SEQUENCE LISTING (24 sequences)

SEQ ID NO: 1 - DNA, 486 bp, Bos taurus
  source 1..486: mol_type = genomic DNA
ctgattgttacccagaccatgaagggtctggacattcaaaaagttgcgggcacctggtacagcctggcgatggcggcgagcgacatcagcctgctggatgcgcagagcgcgccgctgcgtgtgtatgttgaggaactgaagccgaccccggagggcgacctggaaattctgctgcagaaatgggagaacgatgaatgcgcgcagaagaagatcattgcggagaaaaccaagatcccggcggtgttcaagattgacgcgctgaacgaaaacaaagtgctggttctggacaccgattacaaaaagtatctgctgttttgcatggagaacagcgcggagccggaacagagcctggtgtgccaatgcctggttcgtaccccggaagtggacgatgaggcgctggaaaaattcgataaggcgctgaaagcgctgccgatgcacatccgtctgagctttaacccgacccagctggaggaacaatgccacatt

SEQ ID NO: 2 - DNA, 372 bp, Bos taurus
  source 1..372: mol_type = genomic DNA
gaacaattgactaagtgtgaagtttttagagagttgaaggatttgaaaggttacggtggtgtttctttgccagagtgggtttgtactactttccatacttctggttacgatactcaagctatcgttcaaaacaacgattctactgaatacggtttgttccaaattaacaataagatttggtgtaaagatgatcaaaaccctcactcttctaacatttgtaacatctcttgtgataagttcttggatgatgatttgactgatgatattatgtgtgttaagaaaattttggataaggttggtattaattactggttggctcataaggctttgtgttctgaaaaattggatcaatggttgtgtgagaaattgtaa

SEQ ID NO: 3 - DNA, 510 bp, Bos taurus
  source 1..510: mol_type = genomic DNA
caagaacaaaaccaagagcaaccaatcagatgtgaaaaggatgagagatttttctctgataagatcgctaagtacatcccaatccaatatgttttgtctagatacccttcttacggtttgaattactaccaacaaaagcctgttgctttgattaacaaccaattcttgccatacccttactatgctaaaccagctgctgttagatcccctgctcaaattttgcaatggcaagttttgtctaacactgttccagcaaagtcttgtcaagctcaacctactactatggctagacatccacaccctcatttgtctttcatggctattccacctaagaaaaaccaagataagactgaaatcccaactattaatactattgcttctggagagccaacttctactcctactattgaagctgttgagtctactgttgctactttggaggcttctcctgaagttattgagtctccacctgagattaatactgttcaagttacttctactgctgtttaa

SEQ ID NO: 4 - DNA, 600 bp, Bos taurus
  source 1..600: mol_type = genomic DNA
agaccaaaacatccaattaaacatcaaggtttgccacaagaagttttgaacgaaaatttgttgagatttttcgttgctccatttccagaagttttcggtaaagaaaaggttaacgaattgtcaaaagatattggttcagaatctactgaagatcaagctatggaagatatcaagcaaatggaagcagaatcaatttcttcatctgaagaaatcgttccaaattctgttgaacaaaagcatatccaaaaggaagatgttccatctgaaagatatttgggttacttagaacaattgttgagattgaagaaatacaaggttccacaattggaaatcgttccaaattcagctgaagaaagattgcattctatgaaagaaggtattcatgcacaacaaaaagaaccaatgatcggtgttaaccaagaattggcatacttctacccagaattgtttagacaattttatcaattggatgcttacccatctggtgcatggtattacgttccattaggtacacaatacactgatgctccatcattttctgatatcccaaacccaatcggttcagaaaattctgaaaagactactatgccattgtggtaa

SEQ ID NO: 5 - DNA, 624 bp, Bos taurus
  source 1..624: mol_type = genomic DNA
aagaacactatggaacatgtttcttcttctgaagagtctatcatctctcaagaaacttacaagcaagagaaaaacatggctattaatccatctaaggaaaatttgtgttctactttctgtaaggaggttgttagaaacgctaatgaagaggaatactctattggttcttcttctgaggaatctgctgaagttgctactgaggaagttaagattactgttgatgataagcactatcaaaaggctttgaacgagattaatcaattctaccaaaagttccctcaatatttgcaatacttgtatcaaggtccaattgttttgaacccttgggatcaagttaagagaaacgctgttccaatcactcctactttgaacagagaacaattgtctacttctgaggaaaattctaagaaaactgttgatatggaatctactgaggttttcactaagaaaactaagttgactgaggaagagaaaaacagattgaactttttgaagaagatttctcaaagataccaaaagttcgctttgccacaatacttgaagactgtttaccaacatcaaaaggctatgaaaccttggattcaacctaagactaaagttattccttacgttagatatttgtaa

SEQ ID NO: 6 - DNA, 630 bp, Bos taurus
  source 1..630: mol_type = genomic DNA
agagaattggaagagttgaacgttccaggtgaaattgttgaatctttgtcttcttctgaagagtctatcactagaattaataagaaaattgagaagtttcaatctgaagagcaacaacaaactgaagatgagttgcaagataagatccatccattcgctcaaactcaatctttggtttacccattccctggtccaattcctaactctttgcctcaaaatattccacctttgactcaaactccagttgttgttccacctttcttgcaacctgaagttatgggtgtttctaaggttaaagaagctatggctccaaagcataaagagatgccatttcctaagtatccagttgaacctttcactgagtctcaatctttgactttgactgatgttgagaacttgcacttgccattgcctttgttgcaatcttggatgcatcaaccacaccaacctttgccacctactgttatgtttccacctcaatctgttttgtctttgtctcaatctaaggttttgccagttcctcaaaaagctgttccataccctcaaagagatatgccaattcaagcctttttgttgtatcaagaaccagttttgggtcctgttagaggtccattccctattattgtttaa

SEQ ID NO: 7 - DNA, 1156 bp, Gallus gallus
  source 1..1156: mol_type = genomic DNA
atgggttcaattggagctgcatctatggagttctgcttcgacgtcttcaaggagttgaaagtacaccatgccaacgaaaatatcttctattgccccattgctattatgtctgcctagctatggtatatttaggcgcaaaggatctacaagaacgcaaattaataaagttgttaggtttgataagttaccaggtttcggcgactccattgaggcccagtgcggtacttcagttaatgtccattcatcattaagggacatcttgaaccagattactaaacctaatgacgtgtattctttctctttagcaagtaggctgtacgccgaggagaggtatcctatcttacccgaatatttacaatgtgtaaaggagttgtaccgtggaggtctggaacccataaattttcagacggcagccgatcaggctagagagctaattaattcctgggtagaatcacaaacgaatggtataattaggaatgttcttcaaccatctagtgttgactcccaaacggccatggttcttgtcaatgcaatagttttcaagggattgtgggagaaggcatttaaggacgaggatacgcaggccatgcccttcagggttactgagcaggaatccaagccagtgcaaatgatgtaccagataggtctatttcgtgtcgcctctatggcttccgagaaaatgaagattcttgagttacccttcgcctccggcactatgtcaatgcttgtactgctacccgatgaggtatccggcttagaacagctagaatcaattatcaattttgagaagctaacagagtggacatcctcaaacgttatggaggaaaggaaaatcaaagtttacttgcctaggatgaagatggaagaaaagtataatctgacttccgtcctaatggctatgggcataaccgatgttttcagttcctccgctaatttgtccggaatatcctccgcagaatccctgaaaatctctcaagccgtccatgccgcacacgcagaaatcaacgaagccggacgtgaggtcgtaggtagtgccgaggctggtgtcgatgcagctagtgtatccgaggaatttagggcagatcaccccttccttttctgcataaagcacatagcaaccaatgcagtactgttcttcggaagatgtgtatccccc

SEQ ID NO: 8 - DNA, 159 bp, Pentadiplandra brazzeana
  source 1..159: mol_type = genomic DNA
gataagtgcaagaaagtctatgaaaactacccagtatccaaatgtcaactggctaaccagtgcaattatgactgcaagcttgacaagcacgcgaggagtggggagtgtttttacgacgagaaacgcaatctacagtgtatttgcgattactgtgaatat

SEQ ID NO: 9 - DNA, 708 bp, Thaumatococcus daniellii
  source 1..708: mol_type = genomic DNA
atggccgccaccacttgcttcttcttcctcttccccttcctcctcctcctcacgctctcccgcgctgccaccttcgagatcgtcaaccgctgctcctacaccgtgtgggcggccgcctccaaaggcgacgccgccctggacgccggcggccgccagctcaactcgggagagtcctggaccatcaacgtagaacccggcaccaacggtggcaaaatctgggcccgcaccgactgctatttcgacgacagcggcagcggcatctgcaagaccggcgactgcggcggcctcctccggtgcaagcgcttcggccggccgcccaccacgctggcggagttctcgctcaaccagtacggcaaggactacatcgacatctccaacatcaaaggcttcaacgtgccgatggacttcagcccgaccacgcgcggctgccgcggggtgcggtgcgccgccgacatcgtggggcagtgcccggcgaagctgaaggcgccggggggtggttgcaacgatgcgtgcaccgtgttccagacgagcgagtactgctgcaccacggggaagtgcgggccgacggagtactcgcgcttcttcaagaggctttgcccggacgcgttcagttatgtcctggacaagccaaccaccgtcacctgccccggcagctccaactacagggtcactttctgccctactgcccttgaacttgaagacgagtaa

SEQ ID NO: 10 - DNA, 293 bp, Dioscoreophyllum cumminsii
  source 1..293: mol_type = genomic DNA
tgggagagtgggaaattatcgatattggtcccttcacccaaaacttgggaaagtttgctgttgacgaggaaaataaaatcggacagtacggaagactcactttcaacaaggtcattaggccttgtatgaagaaaacaatctacgagaacgaaggctttagagagattaaggggtacgaatatcaactgtacgtgtatgcttccgataaacttttcagagccgatatctctgaggactacaagactagaggcaggaaattgctcaggtttaacggacctgttcctccaccgtag

SEQ ID NO: 11 - DNA, 759 bp, Synsepalum dulcificum
  source 1..759: mol_type = genomic DNA
acaacaatgaaggaattaacaatgctctctctctcgttcttcttcgtctctgcattgttggcagcagcggccaacccactgcttagtgcagcggattcggcacccaatccggttcttgacatagacggagagaaactccggacggggaccaattattacattgtgccggtgctccgcgaccatggcggcggccttacagtatccgccaccacccccaacggcaccttcgtttgtccacccagagttgtccaaacacgaaaggaggtcgaccacgatcgccccctcgctttctttccagagaacccaaaggaagacgttgttcgagtctccaccgatctcaacatcaatttctcggcgttcatgccctgtcgttggaccagttccaccgtgtggcggctcgacaaatacgatgaatccacggggcagtacttcgtgaccatcggcggtgtcaaaggaaacccaggtcccgaaaccattagtagctggtttaagattgaggagttttgtggtagtggtttttacaagcttgttttctgtcccaccgtttgtggttcctgcaaagtaaaatgcggagatgtgggcatttacattgatcagaagggaagaaggcgtttggctctcagcgataaaccattcgcattcgagttcaacaaaaccgtatacttctaattgggtttgggggtggtttttccaatcacatctcatgtatgatcagctccattatcgatctgcataattataattaataaggaagctttt

SEQ ID NO: 12 - DNA, 477 bp, Curculigo latifolia
  source 1..477: mol_type = genomic DNA
atggcggccaagtttcttctcaccattcttgtcacctttgcggccgtcgctagccttggcatggccgacagtgtcctgctctccgggcaaactctgtatgcgggccactccctcacgtcgggcagctataccttaaccatacaaaacaactgcaacctggtgaaataccagcacgggaggcagatctgggctagcgacactgacgggcagggctcccaatgccgcctcacattgcggagtgacgggaacctcattatctacgacgacaacaacatggtcgtgtgggggagcgactgctgggggaacaacggcacgtatgctcttgttcttcagcaggatggcctctttgtcatctatggcccggttttgtggccccttggccttaatgggtgccgcagtcttaatggtgaaatcacagttgctaaggattctactgaaccacaacatgaggatattaagatggtgattaataattaa

SEQ ID NO: 13 - DNA, 590 bp, Curculigo latifolia
  source 1..590: mol_type = genomic DNA
atggcggccaagtttcttctcaccattcttgtcacctttgcggccgtcgctagccttggcatggccgacagtgtcctgctctccgggcaaactctgtatgccggccactccctcacgtcgggcagctataccttaaccatacaaaacaactgcaacctggtgaaataccagcacgggaggcagatctgggctagcgacactgacgggcagggctcccaatgccgcctcacattgcggagtgacgggaacctcattatctacgacgacaacaacatggtcgtgtgggggagcgactgctgggggaacaacggcacgtatgctcttgttcttcagcaggatggcctctttgtcatctatggcccggttttgtggccccttggccttaatgggtgccgcagtcttaatggtgaaatcacagttgctaaggattctactgaaccacaacatgaggatattaagatggtgattaataattaatcaagtgagaggattgttatgagaataatgagggaatggaagaccaatctcatgtcggtgtggcctatctcgacctgtttgcagtgcctttgttaaaataacacattgcttaa

SEQ ID NO: 14 - DNA, 18 bp, synthetic construct
  source 1..18: mol_type = other DNA
  repeat_region 1..9: note = 1..9 can be repeated 1 to 10 times
tctggtgggagcgcgggt

SEQ ID NO: 15 - DNA, 21 bp, synthetic construct
  source 1..21: mol_type = other DNA
  repeat_region 4..18: note = 4..18 can be repeated 1 to 10 times
gctgaagcagctgcgaaagca

SEQ ID NO: 16 - DNA, 48 bp, synthetic construct
  source 1..48: mol_type = other DNA
  repeat_region 13..21: note = 13..21 can be repeated 1 to 10 times
aaaaaaaaaagatctggtgggagcgcgggtaaaagagaggctgaagct

SEQ ID NO: 17 - DNA, 24 bp, synthetic construct
  source 1..24: mol_type = other DNA
  repeat_region 1..12: note = 1..12 can be repeated 1 to 10 times
  repeat_region 13..24: note = 13..24 can be repeated 1 to 10 times
aaaaaaaaaagagaggctgaagct

SEQ ID NO: 18 - DNA, 12 bp, synthetic construct
  source 1..12: mol_type = other DNA
  repeat_region 1..3: note = 1..3 can be repeated 1 to 10 times
gggggggggggg

SEQ ID NO: 19 - DNA, 15 bp, synthetic construct
  source 1..15: mol_type = other DNA
  repeat_region 1..15: note = 1..15 can be repeated 1 to 10 times
gggggtgggggttct

SEQ ID NO: 20 - DNA, 48 bp, synthetic construct
  source 1..48: mol_type = other DNA
  repeat_region 4..18: note = 4..18 is repeated 4 times
  repeat_region 31..45: note = 31..45 is repeated 4 times
gctgaagcagctgcgaaagctttggaagctgaagcagctgcgaaagct

SEQ ID NO: 21 - DNA, 15 bp, synthetic construct
  source 1..15: mol_type = other DNA
  repeat_region 1..6: note = 1..6 can be repeated from 1 to 10 times
ccagctccagctcca

SEQ ID NO: 22 - DNA, 267 bp, Saccharomyces cerevisiae
  source 1..267: mol_type = genomic DNA
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

SEQ ID NO: 23 - DNA, 54 bp, Homo sapiens
  source 1..54: mol_type = genomic DNA
atgaagtgggtcaccttcatttccttgctgttcttgttctcttccgcttactct

SEQ ID NO: 24 - DNA, 276 bp, synthetic construct
  source 1..276: mol_type = other DNA
atgaggcaggtttggttctcttggattgtgggattgttcctatgttttttcaacgtgtcttctgctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

(19) United States
(12) Patent Application Publication
(10) Pub. No.: US 2026/0008825 A1
(43) Pub. Date: Jan. 8, 2026

(54) A NOVEL PROTEIN COMPOSITION AND THEIR USE IN FORMULATING DAIRY PRODUCTS

(71) Applicant: PHYX44 LABS PRIVATE LIMITED, Bangalore, Karnataka (IN)

(72) Inventors: PIYUSH SETHIA, Mumbai, Maharashtra (IN); Bharath BAKARAJU, Bangalore (IN); Vanita UPPADA, Hyderabad (IN); Jayesh Suman VARAVADERKAR, Thane, Maharashtra (IN); Jyotsana PATEL, Jabalpur, Madhya Pradesh (IN); Sambhaji Balkrushna CHAVAN, Pune (IN)

(21) Appl. No.: 18/844,679

(22) PCT Filed: Mar. 9, 2023
(86) PCT No.: PCT/SG2023/050150

§ 371 Date: Sep. 6, 2024

(30) Foreign Application Priority Data
Mar. 9, 2022 (IN) 10202202395T

(51) Int. Cl. C07K 14/43; C07K 14/47; C12N 15/81

(52) U.S. Cl. CPC C07K 14/43; C07K 14/4717; C12N 15/815; C07K 2319/00

(57) ABSTRACT

This invention discloses a milk protein containing composition comprising: at least one milk protein; at least a first protein; at least a second protein; and at least a linker comprising an amino acid sequence coded by nucleotide sequence which is at least 80% similar to sequences selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21

### FIELD OF THE INVENTION

The present invention, described herein, generally relates to a novel protein composition which can be used in formulating dairy products. Particularly, the present invention relates to a recombinant fusion protein, and a method for producing such recombinant protein, which has a sweet taste, is palatable and can be used as an animal-free dairy substitute/s.

### REFERENCE TO SEQUENCE LISTING

The present application is being filed along with a Sequence Listing in electronic format. The sequence listing filed, entitled Sequence.ST25.txt, was created on 09th March 2023 and having a size of 12,288 bytes. The information in electronic format of the Sequence Listing is incorporated herein by reference in its entirety.

### BACKGROUND AND INTRODUCTION

Milk is considered as a rich source of nutrients and is usually desired to be consumed along with sugars in many of the milk-based beverages and other food items. The global population is estimated to exceed 9 billion consumers by 2050 (United nations, 2013) and so would be the increase in demand for nutrition of which dairy constitutes a major component.

As there is an increase in demand for dairy products there is an adverse environmental effect caused by the dairy industry (along with other animal protein generating industries). Vegan plant protein-based alternatives to dairy products in most cases do not meet the protein requirement to the similar extent (Alcorta et al, 2021).

Parallely, sugars are added to several milk, milk-based beverages or other food items usually to impart sweet taste and desired aroma. While milk is considered a rich source of nutrients, there is a growing concern worldwide about co-consuming carbohydrate sugars that are worried to cause obesity, diabetes, caries and/or hyperlipemia. Some of the naturally occurring sugars in natural dairy such as lactose are responsible for intolerances such as lactose intolerance.

Over the past decade, an increased tendency to use artificial low-calorie sweeteners like Saccharin, Aspartame, Cyclamate, Sucralose, Neotame, Acesulfame Potassium, etc. as alternatives has been widely observed. However, even these artificial sweeteners have been proven to have certain adverse health effects such as dizziness, headaches, psychological problems, gastrointestinal issues, and mood changes. This has led to a huge demand for naturally identified and obtained sweet-tasting proteins.

Taste receptors, on the tongue, identify perfectly fitting molecules with a particular structure (which is common to carbohydrate sugars, artificial low-calorie sweeteners and sweet-tasting proteins) and send signals to the brain indicating it to identify it as a ‘Sweet’ tasting compound.

Table sugar sucrose is considered to have a sweetness perception rating of 1 as per the standard convention, and other sweet-tasting substances are rated relative to this. Largely of interest among sweet-tasting proteins are Thaumatin, Monellin and Brazzein that are rated to be 3000, 3000, and 2000 times sweeter than sucrose (Ming and Hellekant, 1994). The massive sweetening capacity of these proteins or similar structure proteins or proteins with similar properties also emphasizes their potential impact when used appropriately.

These concerns have fueled development of fusion proteins that are alternative to milk proteins and carbohydrates and confer nutritional and clinical benefits.

Accordingly, it is envisaged to express, via recombinant expression of purported gene sequences in a microbial system, fusion protein in which one or more protein is fused along with one or more sweet-tasting protein using one or more linkers in a chain or in tandem. The fusion protein would serve not only as a rich source of protein but also as low-calorie non-carbohydrate sweeteners overcoming the otherwise associated health concerns.

### SUMMARY OF THE INVENTION

Even though the present subject matter relates to a protein composition consisting of fused protein, it is to be understood that this application is not limited to a particular composition described, as there can be multiple possible embodiments which are not expressly illustrated in the present disclosure. It is also to be understood that the terminology used in the description is for the purpose of describing implementations or versions or embodiments only and is not intended to limit the scope of the present subject matter.

This summary is provided to introduce aspects related to a protein composition consisting of fused protein. This summary is not intended to identify essential features of the claimed subject matter nor is it intended for use in determining or limiting the scope of the present subject matter.

The present invention is based on the finding that constructing fusion genes with components encoding one or more proteins present in milk along with one or more sweet-tasting proteins that have naturally originated from plants would result in fusion protein molecules that would serve not only as rich source of proteins but also as low-calorie sweet tasting protein thereby overcoming the otherwise associated health concerns posed by alternative sweeteners being used currently. The resulting composition thereby not only preserves native nutritive properties of targeted dairy-based beverages and food items but also imparts desired taste and/or aroma, stability and solubility, while ensuring to avoid possible health concerns that currently prevail worldwide. The milk protein part of the resultant fused protein would serve the same nutritive and functional properties as served by the animal-derived milk protein and also act as a substitute for all of its applications and products derived thereupon. Furthermore, the sweet-tasting protein part of the resultant fused protein would serve to replace/reduce lactose or other sugars in dairy applications and products derived thereupon while increasing the overall protein content of such products.

In one embodiment, the present invention provides a recombinant fusion protein comprising: a) at least a first protein; b) at least a second protein; and c) at least a linker comprising an amino acid sequence coded by the nucleotide sequence which is 80% similar to sequences selected from the group consisting of SEQ ID NO: 3, SEQ ID NO: 4, SEQ ID NO: 5, SEQ ID NO: 6, SEQ ID NO.: 7, SEQ ID NO.: 8, SEQ ID NO.: 9, and SEQ ID NO.: 10

In one embodiment, the present invention provides a recombinant fusion protein, comprising: at least a first protein comprising an amino acid sequence having at least 80% sequence identity to a polypeptide as encoded by a nucleotide sequence selected from the group consisting of SEQ ID NO: 1, SEQ ID NO: 2, SEQ ID NO: 3. SEQ ID NO: 4, SEQ ID NO: 5 SEQ ID NO: 6, and SEQ ID NO: 7; at least a second protein comprising an amino acid sequence having at least 80% sequence identity to a polypeptide as encoded by a nucleotide sequence selected from the group consisting of SEQ ID NO: 8, SEQ ID NO: 9, SEQ ID NO.: 10, SEQ ID NO.: 11, SEQ ID NO.: 12, and SEQ ID NO.: 13; and at least a linker comprising an amino acid sequence coded by the nucleotide sequences which is 80% similar to sequences selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21.

In one embodiment, the present invention provides a recombinant fusion protein, comprising: at least a first protein comprising an amino acid sequence having at least 80% sequence identity to a polypeptide as encoded by the nucleotide sequences set forth in SEQ ID NO: 1; at least a second protein comprising an amino acid sequence having at least 80% sequence identity to the polypeptide as encoded by the nucleotide sequences set forth in SEQ ID NO: 2; and at least a linker comprising an amino acid sequence coded by the nucleotide sequences which is 80% similar to sequences selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21.

In at least an embodiment, the linker is an inert peptide linker.

In one embodiment, the present invention provides a recombinant fusion protein, comprising: a first protein comprising an amino acid sequence having at least 90% sequence identity to the polypeptide as encoded by the nucleotide sequences set forth in SEQ ID NO: 1; a second protein comprising an amino acid sequence having at least 90% sequence identity to the polypeptide as encoded by the nucleotide sequences set forth in SEQ ID NO: 2; and a linker comprising an amino acid sequence coded by nucleotide sequence which is at least 80% similar to sequences selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21.

In one embodiment, the present invention provides a recombinant fusion protein, comprising: a first protein comprising an amino acid sequence having at least 95% sequence identity to the polypeptide as encoded by the nucleotide sequences set forth in SEQ ID NO: 1; a second protein comprising an amino acid sequence having at least 95% sequence identity to the polypeptide as encoded by the nucleotide sequences as set forth in SEQ ID NO: 2; and a linker comprising an amino acid sequence coded by nucleotide sequence which is at least 80% similar to sequences selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21.

In one embodiment, the linker is an inert peptide linker and consists of repetitive sequences of amino acids.

In one embodiment, the first protein is selected from the group comprising of a milk protein, a non-milk bovine protein, and a non-bovine protein.

In one embodiment, the milk protein is a recombinantly expressed protein selected from the group comprising of alpha-lactalbumin, beta-lactoglobulin, kappa-casein, alpha-casein, beta-casein, lactoferrin and the like.

In an exemplary embodiment, the milk protein is a recombinant beta-lactoglobulin.

In one embodiment, the second protein is selected from the group comprising of a low-calorie non-carbohydrate sweetener or a non-milk bovine protein or a non-bovine protein of microbial, animal, plant, or recombinant origin.

In one embodiment, the low-calorie non-carbohydrate sweetener is selected from the group consisting of Brazzein, Thaumatin, Monellin, Curculin, Mabinlin, Miraculin, Neoculin, Pentadin and a combination thereof.

In an exemplary embodiment, the low-calorie sweetener is Brazzein.

In one embodiment, the milk proteins and low-calorie non-carbohydrate sweetener are produced in microbial systems.

In one embodiment, the recombinant fusion protein comprises any combination of the listed one or more first protein and one or more second protein from any origin using one or more linkers.

In one embodiment, the fusion protein comprises any combination of the listed one or more first protein and one or more second protein from any origin, in both orientation, using one or more linkers.

In one embodiment, the fusion protein comprises at least one milk protein; at least one low-calorie sweet tasting protein; and at least one inert peptide linker.

In another embodiment, at least one milk protein is fused with the low-calorie non-carbohydrate sweet tasting protein using an inert peptide linker.

In another embodiment, the fusion protein comprises at least one non-milk bovine protein; at least one low-calorie sweet tasting protein; and at least one inert peptide linker.

In another embodiment, at least one non-milk bovine protein is fused with the low-calorie non-carbohydrate sweetener using an inert peptide linker.

In another embodiment, the fusion protein comprises at least one non-milk non-bovine protein; at least one low-calorie sweet tasting protein; and at least one inert peptide linker.

In another embodiment, at least one non-milk non-bovine protein is fused with the low-calorie non-carbohydrate sweetener using an inert peptide linker.

In an exemplary embodiment, the fusion protein comprising milk protein beta-lactoglobulin is fused with Brazzein using a flexible 12 amino acid chain inert linked peptide.

In another embodiment, the inert peptide linker is selected from flexible linkers or rigid linkers or combination thereof.

In another embodiment, the flexible peptide linkers are of (SGG)ₙSAG nature, wherein n=1-10.

In another embodiment, the flexible peptide linkers are of (G)ₙ nature, wherein n=3-10.

In another embodiment, the flexible peptide linkers are of (GGGGS), nature, wherein n=1-10.

In another embodiment, the rigid peptide linkers are of A(EAAAK)nA nature, wherein n=1-10.

In another embodiment, the linkers are a combination of one or more flexible and rigid inert linker peptide chains mentioned above. The peptide linkers may be a combination of two or more flexible and/or rigid linkers, mentioned above. For instance, in an embodiment, the linker may be [A(EAAAK)nA], or [A(EAAAK)nAA(EAAAK)nA], or [A(EAAAK)nAA(EAAAK)nAA(EAAAK)nA], or [A(EAAAK)nAA(EAAAK)nAA(EAAAK)nAA(EAAAK)nA], or [A(EAAAK)nAA(EAAAK)nAA(EAAAK)nAA(EAAAK)nAA(EAAAK)nA], wherein n=1-10, or n=1-5, or n=3.

In another embodiment, the peptide linkers are of (SGG)ₙSAG-A(EAAAK)ₙA-(GGGGS), nature, wherein n=1-10.

In another embodiment, the first protein and second protein are joined by the inert linker in the reverse orientation.

In an exemplary embodiment, the fusion protein comprising milk protein beta-lactoglobulin is fused with Brazzein using a rigid 17 amino acid chain inert peptide linker

In one embodiment, there is provided a recombinant fusion protein comprising at least one first protein; at least one second protein; and at least one inert peptide linker, wherein the at least one first protein is fused with the second protein using an inert peptide linker.

In another aspect, provided herein is a nucleic acid molecule encoding the recombinant fusion protein described herein.

In another aspect, provided herein is an expression vector, comprising the nucleic acid sequence encoding the recombinant fusion protein described herein.

In some embodiments, the milk proteins and sweet tasting proteins are produced in the microbial systems.

In another embodiment, a bovine dairy protein or non-bovine protein are conjugated to a sweet tasting protein via an inert linker.

In another aspect, provided herein is a composition comprising:
a) at least one recombinant fusion protein in the range of 0.1% to 8%;
b) at least one non-fusion protein in the range of 1% to 10%; and
c) at least one additive in the range of 0.1% to 5%,
wherein, at least one recombinant fusion protein comprises a first protein, a second protein, and a linker having an amino acid sequence coded by nucleotide sequence which is 80% similar to sequences selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21.

In one embodiment, the at least one non-fusion protein is selected from the group consisting of microbial, animal, plant origin or recombinantly expressed protein.

In one embodiment, the recombinant protein is mixed with at least one additive selected from a group consisting of carbohydrates, lipids, fats, ash, micronutrients, vitamins, and the like; in order to obtain a dairy alternative product with a similar mouth feel.

In one embodiment, the fats are of plant origin, microbially derived, fermentation derived or similar.

In one embodiment, plant origin oils are selected from a group consisting of Sunflower oil, corn oil, soybean oil, palm fruit oil, palm kernel oil, safflower oil, flaxseed oil, rice bran oil, cotton seed oil, olive oil, canola oil, flaxseed oil, coconut oil, algal oil, and microbial produced fats.

In one embodiment, the fats are derived from fermentation of plant origin oil substrates using microbes.

In one embodiment, the recombinant composition comprises a fusion protein in which three proteins are linked with two linkers.

In one embodiment, the recombinant fusion protein or the composition is used in formulating dairy and related products.

In one embodiment, the recombinant fusion protein is mixed with plant-based fats, ash, and water to obtain a dairy alternative beverage with a palatable taste.

In one embodiment, the recombinant fusion protein can be used in other dairy products such as Ice-creams, frozen desserts, cakes, coffee creamer.

In one embodiment, the recombinant fusion protein can be used in but not restricted to vegan protein supplements. For instance, the recombinant fusion protein can be used in beverages such as carbonated drinks, fruit juices, liqueurs, and drink powders.

In one aspect, provided herein is a method for producing the recombinant fusion protein comprising fermenting a recombinant host cell capable of producing said recombinant fusion protein.

Additional aspects, advantages, features and objects of the present invention would be made apparent from the detailed description of the illustrative embodiments. It will be appreciated that features of the present invention are susceptible to being combined in various combinations without departing from the scope of the present invention as defined by the below mentioned detailed description.

### BRIEF DESCRIPTION OF THE ACCOMPANYING DRAWINGS

In order to provide an understanding of the embodiments of the invention, reference is made to the appended drawings, which are not necessarily drawn to scale and in which reference numerals refers to components of exemplary embodiments of the invention. The above and other features of the presently claimed invention, their nature and various advantages will become more apparent upon consideration of the following detailed description, taken in conjunction with the accompanying drawings:

FIGS. 1A and 1B illustrates a fusion of a representative protein A and protein B using a representative peptide linkage C in accordance with one embodiment of the present invention;

FIG. 2 is a vector map of a integrative vector used for production of fusion proteins provided herein, in accordance with representative embodiments of the present invention;

FIGS. 3A-3D is an example of various gene cassette representation, in accordance with representative embodiments of the present invention;

FIG. 4 is a gel image for expression of recombinant fusion protein Beta lactoglobulin-L1-Brazzein, Brazzein-L1-beta lactoglobulin, Beta lactoglobulin-L2-Brazzein, Brazzein-L2-beta lactoglobulin; and

FIG. 5 is a gel image for expression of recombinant fusion protein Beta lactoglobulin-L3-Brazzein, Beta lactoglobulin-L4-Brazzein.

### DETAILED DESCRIPTION OF THE INVENTION

The invention will now be described with reference to the accompanying embodiments which do not limit the scope and ambit of the invention. The description provided is purely by way of example and illustration.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Some embodiments of this invention, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described.

In an aspect of the present invention, in order to overcome the problems in prior art and to provide various advantages elaborated in the subsequent section, a protein composition comprising fusion protein for formulating dairy and related product is disclosed.

As used in the specification and claims, the singular forms “a”, “an” and “the” include plural references unless the context clearly dictates otherwise. For example, the term “an article” may include a plurality of articles unless the context clearly dictates otherwise. Those with ordinary skill in the art will appreciate that the elements in the figures are illustrated for simplicity and clarity and are not necessarily drawn to scale. For example, the dimensions of some of the elements in the figures may be exaggerated, relative to other elements, in order to improve the understanding of the present invention. There may be additional components described in the foregoing application that are not depicted on one of the described drawings. In the event such a component is described, but not depicted in a drawing, the absence of such a drawing should not be considered as an omission of such design from the specification.

As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

The present invention is focused on creation of fused proteins using selected proteins and/or low-calorie sweet tasting proteins by using an inert peptide linker to achieve resultant protein with sweetness, stability and solubility, while ensuring to avoid possible health concerns that currently prevail worldwide. Taste receptors on the tongue identify perfectly fitting molecules with a particular structure (common to carbohydrate sugars, artificial low-calorie sweeteners and sweet-tasting proteins) and send signals to the brain indicating it to be sweet in taste. The sweet-tasting proteins are usually ˜50 to 200 amino acids in length possessing molecular weight ˜6.5 to 30 Kda (Zhao et al, 2021).

Accordingly, provided herein is a recombinant fusion protein comprising at least one first protein; at least one second protein; and at least one inert peptide linker, wherein the at least one first protein is fused with the second protein using an inert peptide linker. The disclosure also provides methods for producing the recombinant fusion protein and compositions comprising the same.

Milk is composed of 2 major classes of proteins-caseins (αS1-casein, αS2-casein, β-casein and κ-casein) and whey proteins (β-lactoglobulin and α-lactalbumin). Caseins are known to interact with each other to give rise to sub-micelles and micelles that play a crucial role in stability and nutritious properties of milk. While the αS1-casein, αS2-casein and β-casein molecules remain embedded inside, K-casein protrudes partially out of the micellar structure. Whey proteins remain suspended and do not participate in formation of micelles.

As used herein, a “milk protein” is any protein, or fragment or variant thereof, that is typically found in one or more mammalian milk. In some embodiments, the milk proteins described herein are casein proteins, such as kappa casein, para—kappa—casein, beta—casein, alpha—S1—casein, and alpha—S2—casein.

In an aspect of the present invention, in order to overcome the problems in prior art and to provide various advantages elaborated in the subsequent section, a protein composition comprising fusion protein for formulating dairy and related product is disclosed.

Accordingly, the present invention in one embodiment is focused on creation of fused proteins using selected proteins and/or a low-calorie sweet tasting protein by using an inert peptide linker to achieve one or more of the goals of resultant protein such as sweetness, stability and solubility.

Constructing fusion genes with components encoding one or more proteins present in milk along with one or more sweet-tasting proteins that have naturally originated from plants would result in fusion protein molecules that would then serve not only as rich source of proteins but also as low-calorie sweet tasting protein thereby overcoming the otherwise associated health concerns posed by alternative sweeteners being used currently.

The composition thereby not only preserves native nutritive properties of targeted milk-based beverages and food items but also imparting desired taste and/or aroma, stability and solubility, while ensuring to avoid possible health concerns that currently prevail worldwide.

The milk protein part of the resultant fused protein would serve the same nutritive and functional properties as served by the animal-derived milk protein and also act as an apt substitute for all of its applications and products derived thereupon. Furthermore, the sweet-tasting protein part of the resultant fused protein would serve to replace/reduce lactose or other sugars while increasing the overall protein content of such products.

In the following description, for the purpose of explanation, specific details are set forth in order to provide an understanding of the present invention. It will be apparent, however, to one skilled in the art that the present invention may be practiced without these details. One skilled in the art will recognize that embodiments of the present invention, some of which are described below, may be incorporated into a number of systems.

In view of the above, the present invention discloses a protein composition comprising a fusion protein in which two proteins are linked with one linker.

The composition comprises a fusion protein in which two proteins are linked with one linker. Alternatively, the composition comprises a fusion protein in which three proteins are linked with two linkers. Alternatively, the fusion protein comprises any combination of the listed one or more first protein and one or more second protein from any origin using one or more linker.

Constructing fusion genes with components encoding one or more proteins present in milk along with one or more sweet-tasting proteins that have naturally originated from plants results into the fusion protein molecules. It involves use of an inert peptide linker with desired level of flexibility and stability required during translation and also acts as a bridge connecting the two resulting proteins synthesized as a fusion protein. Typically, the inert peptide linker comprises at least four amino acids that impart one or more of the following: A) flexibility, or B) Rigidity, or C) Improve solubility, or D) Increase stability of the fused protein.

The composition thereby not only preserves native nutritive properties of targeted milk-based beverages and food items but also imparting desired taste and/or aroma, stability and solubility, while ensuring to avoid possible health concerns that currently prevail worldwide. The milk protein part of the resultant fused protein would serve the same nutritive and functional properties as served by the animal-derived milk protein and also act as an apt substitute for all of its applications and products derived thereupon. Furthermore, the sweet-tasting protein part of the resultant fused protein would serve to replace/reduce lactose or other sugars while increasing the overall protein content of such products.

In one exemplary embodiment of the present invention there is provided a composition comprising a fusion protein comprising milk protein Beta-lactoglobulin fused with Brazzein using a flexible 12 amino acid chain inert peptide linker

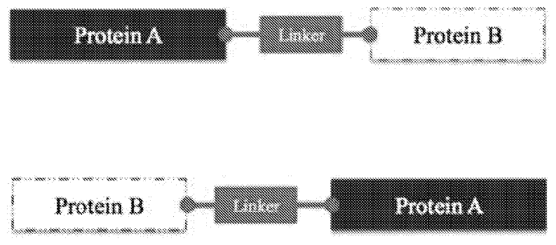

The accompanying FIG. 1A and FIG. 1B illustrates fusion of a representative protein A and protein B using a representative inert peptide linkage in accordance with one embodiment of the present invention. The protein A can be a milk protein or non-milk bovine protein or non-bovine protein of microbial, animal, plant origin or recombinant protein. The protein B is a low-calorie non-carbohydrate sweetener or milk protein or non-milk bovine protein or non-bovine protein of microbial, animal, plant origin or recombinant protein.

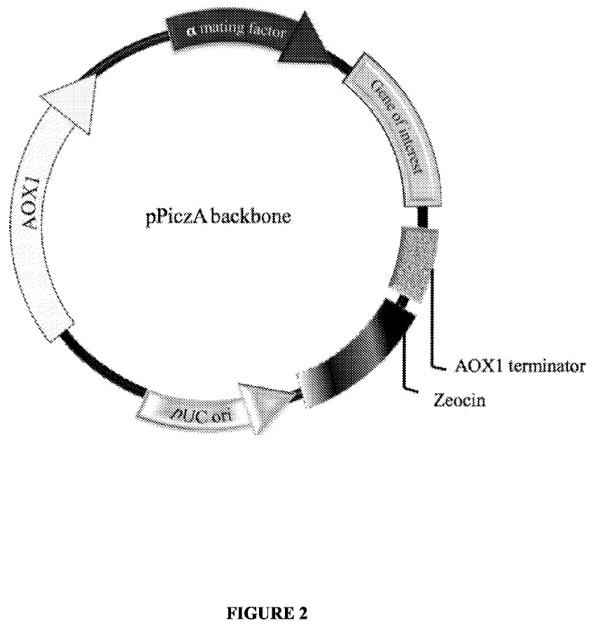

The accompanying FIG. 2 illustrates the example of vector used for driving the expression. According to the embodiment, the genes were cloned in pPICZA, integrative vector which contain Pichia pastoris alcohol oxidase promoter, alcohol oxidase terminator, zeocin resistance gene. Integrative vector containing methanol inducible promoter (AOX1, FLD1, MOX1, DAS1 etc), alpha mating signal factor, zeocin resistance cassette, terminator (illustrated in FIG. 2). Further example of vector used herein is integrative vector containing constitutive promoter, alpha mating signal factor, zeocin resistance cassette, terminator. Similar to the above, wherein zeocin is replaced with kanamycin resistance. Similarly, alpha mating factor is replaced by any signal factor driving the expression to extracellular fraction.

Expression Organisms: The recombinant molecule in the above embodiments can be expressed in microbial systems used for recombinant molecules such as Bacteria-Escherichia coli, Bacillus subtilis, Cyanobacteria etc. Examples of some of the Fungal expression hosts-Aspergillus niger, Aspergillus nidulans, Trichoderma reesei, Pencillum sps. Examples of some of the Yeasts—Saccharomyces cerevisiae, Candida albicans, Kluyveromyces lactis, Kluyveromyces marxianus, Yarrowia sps etc and methylotrophic yeast-Pichia pastoris, Hansenula polymorpha, Candida boidini.

Transformation: Transformation protocols like chemical transformation using Lithium acetate and LiCl, transfection, electroporation, PEG mediated, agrobacterium mediated transformation etc can be used.

Proteins of interest:
Milk proteins:—Some examples of milk proteins used in the present invention includes Kappa casein, alpha-S1-casein, alpha-S2-casein, Beta-casein, beta-lactoglobulin, alpha lactalbumin, transferrin
Other Animal Proteins—some examples of the other proteins in the invention include ovalbumin, ovotransferrin, ovomucoid

### Beta Lactoglobulin

Bovine beta lactoglobulin is a major whey protein of bovine milk which is a part of the few embodiments of the invention. The sequence of the gene is as given below.

[SEQ ID NO: 1]
CTGATTGTTACCCAGACCATGAAGGGTCTGGACATTCAAAAAGTTG
CGGGCACCTGGTACAGCCTGGCGATGGCGGCGAGCGACATCAGCCT
GCTGGATGCGCAGAGCGCGCCGCTGCGTGTGTATGTTGAGGAACTG
AAGCCGACCCCGGAGGGCGACCTGGAAATTCTGCTGCAGAAATGGG
AGAACGATGAATGCGCGCAGAAGAAGATCATTGCGGAGAAAACCA
AGATCCCGGCGGTGTTCAAGATTGACGCGCTGAACGAAAACAAAGT
GCTGGTTCTGGACACCGATTACAAAAAGTATCTGCTGTTTTGCATGG
AGAACAGCGCGGAGCCGGAACAGAGCCTGGTGTGCCAATGCCTGGT
TCGTACCCCGGAAGTGGACGATGAGGCGCTGGAAAAATTCGATAAG
GCGCTGAAAGCGCTGCCGATGCACATCCGTCTGAGCTTTAACCCGA
CCCAGCTGGAGGAACAATGCCACATT

Alpha Lactalbumin Alpha lactalbumin is a whey protein of bovine milk which is a part of the few embodiments of the invention. The sequence of the gene is as given below

[SEQ ID NO: 2]
GAACAATTGACTAAGTGTGAAGTTTTTAGAGAGTTGAAGGATTTGA
AAGGTTACGGTGGTGTTTCTTTGCCAGAGTGGGTTTGTACTACTTTC
CATACTTCTGGTTACGATACTCAAGCTATCGTTCAAAACAACGATTC
TACTGAATACGGTTTGTTCCAAATTAACAATAAGATTTGGTGTAAAG
ATGATCAAAACCCTCACTCTTCTAACATTTGTAACATCTCTTGTGAT
AAGTTCTTGGATGATGATTTGACTGATGATATTATGTGTGTTAAGAA
AATTTTGGATAAGGTTGGTATTAATTACTGGTTGGCTCATAAGGCTT
TGTGTTCTGAAAAATTGGATCAATGGTTGTGTGAGAAATTGTAA

### Kappa Casein

Kappa casein is a major casein of the bovine milk protein which is involved in the stabilization of the milk micelles. It is a part of some embodiments of the invention.

[SEQ ID NO: 3]
CAAGAACAAAACCAAGAGCAACCAATCAGATGTGAAAAGGATGAG
AGATTTTTCTCTGATAAGATCGCTAAGTACATCCCAATCCAATATGT
TTTGTCTAGATACCCTTCTTACGGTTTGAATTACTACCAACAAAAGC
CTGTTGCTTTGATTAACAACCAATTCTTGCCATACCCTTACTATGCTA
AACCAGCTGCTGTTAGATCCCCTGCTCAAATTTTGCAATGGCAAGTT
TTGTCTAACACTGTTCCAGCAAAGTCTTGTCAAGCTCAACCTACTAC
TATGGCTAGACATCCACACCCTCATTTGTCTTTCATGGCTATTCCAC
CTAAGAAAAACCAAGATAAGACTGAAATCCCAACTATTAATACTAT
TGCTTCTGGAGAGCCAACTTCTACTCCTACTATTGAAGCTGTTGAGT
CTACTGTTGCTACTTTGGAGGCTTCTCCTGAAGTTATTGAGTCTCCA
CCTGAGATTAATACTGTTCAAGTTACTTCTACTGCTGTTTAA

### Alpha S1 Casein

Alpha S1 casein is another major casein and is a major component of bovine milk. It is a part of some of the embodiments of the invention.

[SEQ ID NO: 4]
AGACCAAAACATCCAATTAAACATCAAGGTTTGCCACAAGAAGTTT
TGAACGAAAATTTGTTGAGATTTTTCGTTGCTCCATTTCCAGAAGTT
TTCGGTAAAGAAAAGGTTAACGAATTGTCAAAAGATATTGGTTCAG
AATCTACTGAAGATCAAGCTATGGAAGATATCAAGCAAATGGAAGC
AGAATCAATTTCTTCATCTGAAGAAATCGTTCCAAATTCTGTTGAAC
AAAAGCATATCCAAAAGGAAGATGTTCCATCTGAAAGATATTTGGG
TTACTTAGAACAATTGTTGAGATTGAAGAAATACAAGGTTCCACAA
TTGGAAATCGTTCCAAATTCAGCTGAAGAAAGATTGCATTCTATGA
AAGAAGGTATTCATGCACAACAAAAAGAACCAATGATCGGTGTTAA
CCAAGAATTGGCATACTTCTACCCAGAATTGTTTAGACAATTTTATC
AATTGGATGCTTACCCATCTGGTGCATGGTATTACGTTCCATTAGGT
ACACAATACACTGATGCTCCATCATTTTCTGATATCCCAAACCCAAT
CGGTTCAGAAAATTCTGAAAAGACTACTATGCCATTGTGGTAA

### Alpha S2 Casein

Alpha S2 casein is a casein protein and is a component of bovine milk. It is a part of some of the embodiments of the invention.

[SEQ ID NO: 5]
AAGAACACTATGGAACATGTTTCTTCTTCTGAAGAGTCTATCATCTC
TCAAGAAACTTACAAGCAAGAGAAAAACATGGCTATTAATCCATCT
AAGGAAAATTTGTGTTCTACTTTCTGTAAGGAGGTTGTTAGAAACGC
TAATGAAGAGGAATACTCTATTGGTTCTTCTTCTGAGGAATCTGCTG
AAGTTGCTACTGAGGAAGTTAAGATTACTGTTGATGATAAGCACTA
TCAAAAGGCTTTGAACGAGATTAATCAATTCTACCAAAAGTTCCCTC
AATATTTGCAATACTTGTATCAAGGTCCAATTGTTTTGAACCCTTGG
GATCAAGTTAAGAGAAACGCTGTTCCAATCACTCCTACTTTGAACA
GAGAACAATTGTCTACTTCTGAGGAAAATTCTAAGAAAACTGTTGA
TATGGAATCTACTGAGGTTTTCACTAAGAAAACTAAGTTGACTGAG
GAAGAGAAAAACAGATTGAACTTTTTGAAGAAGATTTCTCAAAGAT
ACCAAAAGTTCGCTTTGCCACAATACTTGAAGACTGTTTACCAACAT
CAAAAGGCTATGAAACCTTGGATTCAACCTAAGACTAAAGTTATTC
CTTACGTTAGATATTTGTAA

### Beta Casein

Beta casein is a major casein protein and is a component of bovine milk. It is a part of some of the embodiments of the invention.

[SEQ ID NO: 6]
AGAGAATTGGAAGAGTTGAACGTTCCAGGTGAAATTGTTGAATCTT
TGTCTTCTTCTGAAGAGTCTATCACTAGAATTAATAAGAAAATTGAG
AAGTTTCAATCTGAAGAGCAACAACAAACTGAAGATGAGTTGCAAG
ATAAGATCCATCCATTCGCTCAAACTCAATCTTTGGTTTACCCATTC
CCTGGTCCAATTCCTAACTCTTTGCCTCAAAATATTCCACCTTTGACT
CAAACTCCAGTTGTTGTTCCACCTTTCTTGCAACCTGAAGTTATGGG
TGTTTCTAAGGTTAAAGAAGCTATGGCTCCAAAGCATAAAGAGATG
CCATTTCCTAAGTATCCAGTTGAACCTTTCACTGAGTCTCAATCTTTG
ACTTTGACTGATGTTGAGAACTTGCACTTGCCATTGCCTTTGTTGCA
ATCTTGGATGCATCAACCACACCAACCTTTGCCACCTACTGTTATGT
TTCCACCTCAATCTGTTTTGTCTTTGTCTCAATCTAAGGTTTTGCCAG
TTCCTCAAAAAGCTGTTCCATACCCTCAAAGAGATATGCCAATTCAA
GCCTTTTTGTTGTATCAAGAACCAGTTTTGGGTCCTGTTAGAGGTCC
ATTCCCTATTATTGTTTAA

### Ovalbumin

Ovalbumin is the main protein found in egg white, making up approximately 55% of the total protein. It is a part of some of the embodiments of the invention.

[SEQ ID NO: 7]
ATGGGTTCAATTGGAGCTGCATCTATGGAGTTCTGCTTCGACGTCTT
CAAGGAGTTGAAAGTACACCATGCCAACGAAAATATCTTCTATTGC
CCCATTGCTATTATGTCTGCCTAGCTATGGTATATTTAGGCGCAAAG
GATCTACAAGAACGCAAATTAATAAAGTTGTTAGGTTTGATAAGTT
ACCAGGTTTCGGCGACTCCATTGAGGCCCAGTGCGGTACTTCAGTTA
ATGTCCATTCATCATTAAGGGACATCTTGAACCAGATTACTAAACCT
AATGACGTGTATTCTTTCTCTTTAGCAAGTAGGCTGTACGCCGAGGA
GAGGTATCCTATCTTACCCGAATATTTACAATGTGTAAAGGAGTTGT
ACCGTGGAGGTCTGGAACCCATAAATTTTCAGACGGCAGCCGATCA
GGCTAGAGAGCTAATTAATTCCTGGGTAGAATCACAAACGAATGGT
ATAATTAGGAATGTTCTTCAACCATCTAGTGTTGACTCCCAAACGGC
CATGGTTCTTGTCAATGCAATAGTTTTCAAGGGATTGTGGGAGAAG
GCATTTAAGGACGAGGATACGCAGGCCATGCCCTTCAGGGTTACTG
AGCAGGAATCCAAGCCAGTGCAAATGATGTACCAGATAGGTCTATT
TCGTGTCGCCTCTATGGCTTCCGAGAAAATGAAGATTCTTGAGTTAC
CCTTCGCCTCCGGCACTATGTCAATGCTTGTACTGCTACCCGATGAG
GTATCCGGCTTAGAACAGCTAGAATCAATTATCAATTTTGAGAAGCT
AACAGAGTGGACATCCTCAAACGTTATGGAGGAAAGGAAAATCAA
AGTTTACTTGCCTAGGATGAAGATGGAAGAAAAGTATAATCTGACT
TCCGTCCTAATGGCTATGGGCATAACCGATGTTTTCAGTTCCTCCGC
TAATTTGTCCGGAATATCCTCCGCAGAATCCCTGAAAATCTCTCAAG
CCGTCCATGCCGCACACGCAGAAATCAACGAAGCCGGACGTGAGGT
CGTAGGTAGTGCCGAGGCTGGTGTCGATGCAGCTAGTGTATCCGAG
GAATTTAGGGCAGATCACCCCTTCCTTTTCTGCATAAAGCACATAGC
AACCAATGCAGTACTGTTCTTCGGAAGATGTGTATCCCCC

Sweet tasting proteins: Examples of low-calorie non-carbohydrate sweetener includes Brazzein, Thaumatin, Monellin, Mabinlin, Miraculin, Curculin, Neoculin and Pentadin and like. Brazzein is used herein as low-calorie non-carbohydrate sweetener. Brazzein is a sweet tasting protein from the plant species Pentadiplandra brazzeana and is about 2000 times sweeter than the sucrose on a molar basis. It is found in two forms in nature. The major form starts with a pyroglutamic acid residue and is 54 amino acids long and the minor form starts with the next amino acid Aspartic acid and 53 amino acids long. The minor form was reported to have better sweet tasting capacity therefore, the minor form is selected from the uniprot ID P56552 (sequence is given below), codon optimized for Pichia pastoris and synthesized.

### Brazzein

Brazzein is a sweet tasting protein found in the extracts of the plant Pentadiplandra brazzeana. It tastes 2000 times sweeter than sucrose on a molar basis. It is a part of some of the embodiments of the invention

[SEQ ID NO: 8]
GATAAGTGCAAGAAAGTCTATGAAAACTACCCAGTATCCAAATGTC
AACTGGCTAACCAGTGCAATTATGACTGCAAGCTTGACAAGCACGC
GAGGAGTGGGGAGTGTTTTTACGACGAGAAACGCAATCTACAGTGT
ATTTGCGATTACTGTGAATAT

### Thaumatin

Thaumatin is a sweet protein found in the fruits of Thaumatococcusdaniellii. It is 100000 times sweeter than sucrose on a molar basis. It is a part of some of the embodiments of the invention

[SEQ ID NO: 9]
ATGGCCGCCACCACTTGCTTCTTCTTCCTCTTCCCCTTCCTCCTCCTC
CTCACGCTCTCCCGCGCTGCCACCTTCGAGATCGTCAACCGCTGCTC
CTACACCGTGTGGGCGGCCGCCTCCAAAGGCGACGCCGCCCTGGAC
GCCGGCGGCCGCCAGCTCAACTCGGGAGAGTCCTGGACCATCAACG
TAGAACCCGGCACCAACGGTGGCAAAATCTGGGCCCGCACCGACTG
CTATTTCGACGACAGCGGCAGCGGCATCTGCAAGACCGGCGACTGC
GGCGGCCTCCTCCGGTGCAAGCGCTTCGGCCGGCCGCCCACCACGC
TGGCGGAGTTCTCGCTCAACCAGTACGGCAAGGACTACATCGACAT
CTCCAACATCAAAGGCTTCAACGTGCCGATGGACTTCAGCCCGACC
ACGCGCGGCTGCCGCGGGGTGCGGTGCGCCGCCGACATCGTGGGGC
AGTGCCCGGCGAAGCTGAAGGCGCCGGGGGGTGGTTGCAACGATGC
GTGCACCGTGTTCCAGACGAGCGAGTACTGCTGCACCACGGGGAAG
TGCGGGCCGACGGAGTACTCGCGCTTCTTCAAGAGGCTTTGCCCGG
ACGCGTTCAGTTATGTCCTGGACAAGCCAACCACCGTCACCTGCCCC
GGCAGCTCCAACTACAGGGTCACTTTCTGCCCTACTGCCCTTGAACT
TGAAGACGAGTAA

### Monellin

[SEQ ID NO: 10]
ATGGGAGAGTGGGAAATTATCGATATTGGTCCCTTCACCCAAAACT
TGGGAAAGTTTGCTGTTGACGAGGAAAATAAAATCGGACAGTACGG
AAGACTCACTTTCAACAAGGTCATTAGGCCTTGTATGAAGAAAACA
ATCTACGAGAACGAAGGCTTTAGAGAGATTAAGGGGTACGAATATC
AACTGTACGTGTATGCTTCCGATAAACTTTTCAGAGCCGATATCTCT
GAGGACTACAAGACTAGAGGCAGGAAATTGCTCAGGTTTAACGGAC
CTGTTCCTCCACCGTAG

### Miraculin

[SEQ ID NO: 11]
ACAACAATGAAGGAATTAACAATGCTCTCTCTCTCGTTCTTCTTCGT
CTCTGCATTGTTGGCAGCAGCGGCCAACCCACTGCTTAGTGCAGCG
GATTCGGCACCCAATCCGGTTCTTGACATAGACGGAGAGAAACTCC
GGACGGGGACCAATTATTACATTGTGCCGGTGCTCCGCGACCATGG
CGGCGGCCTTACAGTATCCGCCACCACCCCCAACGGCACCTTCGTTT
GTCCACCCAGAGTTGTCCAAACACGAAAGGAGGTCGACCACGATCG
CCCCCTCGCTTTCTTTCCAGAGAACCCAAAGGAAGACGTTGTTCGAG
TCTCCACCGATCTCAACATCAATTTCTCGGCGTTCATGCCCTGTCGTT
GGACCAGTTCCACCGTGTGGCGGCTCGACAAATACGATGAATCCAC
GGGGCAGTACTTCGTGACCATCGGCGGTGTCAAAGGAAACCCAGGT
CCCGAAACCATTAGTAGCTGGTTTAAGATTGAGGAGTTTTGTGGTAG
TGGTTTTTACAAGCTTGTTTTCTGTCCCACCGTTTGTGGTTCCTGCAA
AGTAAAATGCGGAGATGTGGGCATTTACATTGATCAGAAGGGAAGA
AGGCGTTTGGCTCTCAGCGATAAACCATTCGCATTCGAGTTCAACAA
AACCGTATACTTCTAATTGGGTTTGGGGGTGGTTTTTCCAATCACAT
CTCATGTATGATCAGCTCCATTATCGATCTGCATAATTATAATTAAT
AAGGAAGCTTTT

### Curculin

[SEQ ID NO: 12]
ATGGCGGCCAAGTTTCTTCTCACCATTCTTGTCACCTTTGCGGCCGT
CGCTAGCCTTGGCATGGCCGACAGTGTCCTGCTCTCCGGGCAAACTC
TGTATGCGGGCCACTCCCTCACGTCGGGCAGCTATACCTTAACCATA
CAAAACAACTGCAACCTGGTGAAATACCAGCACGGGAGGCAGATCT
GGGCTAGCGACACTGACGGGCAGGGCTCCCAATGCCGCCTCACATT
GCGGAGTGACGGGAACCTCATTATCTACGACGACAACAACATGGTC
GTGTGGGGGAGCGACTGCTGGGGGAACAACGGCACGTATGCTCTTG
TTCTTCAGCAGGATGGCCTCTTTGTCATCTATGGCCCGGTTTTGTGG
CCCCTTGGCCTTAATGGGTGCCGCAGTCTTAATGGTGAAATCACAGT
TGCTAAGGATTCTACTGAACCACAACATGAGGATATTAAGATGGTG
ATTAATAATTAA

### Neoculin

[SEQ ID NO: 13]
ATGGCGGCCAAGTTTCTTCTCACCATTCTTGTCACCTTTGCGGCCGT
CGCTAGCCTTGGCATGGCCGACAGTGTCCTGCTCTCCGGGCAAACTC
TGTATGCCGGCCACTCCCTCACGTCGGGCAGCTATACCTTAACCATA
CAAAACAACTGCAACCTGGTGAAATACCAGCACGGGAGGCAGATCT
GGGCTAGCGACACTGACGGGCAGGGCTCCCAATGCCGCCTCACATT
GCGGAGTGACGGGAACCTCATTATCTACGACGACAACAACATGGTC
GTGTGGGGGAGCGACTGCTGGGGGAACAACGGCACGTATGCTCTTG
TTCTTCAGCAGGATGGCCTCTTTGTCATCTATGGCCCGGTTTTGTGG
CCCCTTGGCCTTAATGGGTGCCGCAGTCTTAATGGTGAAATCACAGT
TGCTAAGGATTCTACTGAACCACAACATGAGGATATTAAGATGGTG
ATTAATAATTAATCAAGTGAGAGGATTGTTATGAGAATAATGAGGG
AATGGAAGACCAATCTCATGTCGGTGTGGCCTATCTCGACCTGTTTG
CAGTGCCTTTGTTAAAATAACACATTGCTTAA

Linkers: Linkers are peptide molecules which are used to link two protein molecules to improve solubility, stability and/or expression. Drugs like Enbrel®, Ontak®, Orencia®, Amevive®, Arcalyst®, and Nplate® contain multiple proteins connected via linkers and have been approved by the FDA. Linkers can be classified as flexible linkers or rigid linkers (Chen et al., 2013; Smith et al. 2003).

Flexible linkers are predominantly composed of small sized amino acids like Glycine, Alanine and/or Serine which form a disordered loop thus providing relatively higher freedom for folding (Argos, 1990). For example, “L1 Type Linker” used herein is composed of amino acids (SGG)nSAG [corresponding nucleotide sequence is SEQ ID NO: 14].

A noted limitation of flexible linkers is the lack of rigidity. There are examples in literature where use of flexible linkers yielded relatively lower yield/reduced biological activity (Amet et al., 2009; Maeda et al., 1997). Many alpha helix forming linkers have been used in literature to construct recombinant fusion proteins (Amet et al., 2009; Bai and Shen, 2006). For example, “L2 Type Linker” used herein is composed of amino acids A(EAAAK)nA [corresponding nucleotide sequence is SEQ ID NO: 15].

Further modification of flexible and rigid linkers could also be used to achieve required yield/biological activity as have been demonstrated in literature (Chen et al., 2013). For example, “L3 Type Linker” used herein is composed of amino acids KKKR(SGG)ₙSAGKREAEA [corresponding nucleotide sequence is SEQ ID NO: 16] and “L4 Type Linker” used herein is composed of amino acids (KKKR)ₙ(EAEA)ₙ [corresponding nucleotide sequence is SEQ ID NO: 17].

L1 Type Linker
SEQ ID NO: 14
(TCTGGTGGG)ₙAGCGCGGGT
where n = 1-10
L2 Type Linker
SEQ ID NO: 15
GCT(GAAGCAGCTGCGAAA)ₙGCA
Where n = 1-10
L3 Type Linker
SEQ ID NO: 16
AAAAAAAAAAGA(TCTGGTGGG)ₙAGCGCGGGTAAAAGA
GAGGCTGAAGCT
Where n = 1-10
L4 Type Linker
SEQ ID NO: 17
(AAAAAAAAAAGA)ₙ(GAGGCTGAAGCT)ₙ
Where n = 1-10
L5 type linker
SEQ ID NO: 18
(GGG)ₙ
Where n = 3-10
L6 type linker
SEQ ID NO: 19
(GGGGGTGGGGGTTCT)ₙ
Where n = 1-10
L7 type linker
SEQ ID NO: 20
GCT(GAAGCAGCTGCGAAA)₄GCTTTGGAAGCT(GAAGCAGC
TGCGAAA)₄GCT
L8 type linker
SEQ ID NO: 21
(CCAGCT)ₙCCA
n = 3-10

As mentioned above, the fusion protein comprises any combination of the listed one or more first protein and one or more second protein from any origin using one or more linkers. The linkers may be a combination of two or more flexible and/or rigid linkers, mentioned above. For instance, in an embodiment, the linker may be -L2-L2-, or -L2-L2-L2-, or -L2-L2-L2-L2-, or -L2-L2-L2-L2-L2-. Herein, L2 refers to “L2 Type Linker” composed of amino acids A(EAAAK)nA, wherein n=1-10, or n=1-5, or n=3. Said otherwise, the linker may be [A(EAAAK)nAA(EAAAK)nA], or [A(EAAAK)nAA(EAAAK)nAA(EAAAK)nA], or [A(EAAAK)nAA(EAAAK)nAA(EAAAK)nAA(EAAAK)nA], or [A(EAAAK)nAA(EAAAK)nAA(EAAAK)nAA(EAAAK)nAA(EAAAK)nA], wherein n=1-10, or n=1-5, or n=3.

As evident from above mentioned linkers, they consist of repetitive sequences of amino acids. This particular repetitive sequence can be repeated “n” number of times to alter the length and property of the linker sequence as per the requirement, wherein “n” could range from 1 to 10. Optimal number of repeats is essential to maintain the functionality and stability of the protein. If the proteins are too close it might affect the 3D structure and hence the functionality. If they are too far, stability of the recombinant molecule is affected. Brazzein protein has four intra-disulfide bridges Cys4-Cys52, Cys16-Cys37, Cys22-Cys47, and Cys26-Cys49. These intra-disulfide bridges make brazzein as the most thermostable sweet protein. This property is important as most of the artificial sweeteners cannot withstand the high temperatures used in baking and other cooking practices. As is evident one of the di-sulfide bridges is close to both the N-terminal and C-terminal hence a very short linker of just one or two of the repeat motif will cause a steric hindrance from the first protein for the correct conformation of the brazzein protein to be stabilized by the Cys4-Cys52 di-sulfide bridges. This would be applicable for both orientations of the recombinant molecule. Accordingly in our example we have used n=3 which maintains an appropriate separation so as to not affect the di-sulfide bridge formation and hence the structure of Brazzein protein.

Vectors: The genes were cloned in pPICZA, integrative vectors which contain Pichia pastoris alcohol oxidase promoter, Alcohol oxidase terminator, Zeocin resistance gene.

Signal peptides: Alpha mating signal factor-Yeast alpha mating factor's pro region is a secretory signal factor which has been extensively used in expression of recombinant proteins in extracellular fractions. In few of the embodiments the signal factor which has been optimized for extracellular secretion in Pichia pastoris has been used. The sequence is provided below:

SEQ ID NO: 22
ATGAGATTTCCTTCAATTTTTACTGCTGTTTTATTCGCAGCATCCTCC
GCATTAGCTGCTCCAGTCAACACTACAACAGAAGATGAAACGGCAC
AAATTCCGGCTGAAGCTGTCATCGGTTACTCAGATTTAGAAGGGGA
TTTCGATGTTGCTGTTTTGCCATTTTCCAACAGCACAAATAACGGGT
TATTGTTTATAAATACTACTATTGCCAGCATTGCTGCTAAAGAAGAA
GGGGTATCTCTCGAGAAAAGAGAGGCTGAAGCT

### Human Serum Albumin Signal Factor

Human serum albumin signal factor is another commonly used signal peptide for protein expression in Pichia pastoris. The signal factor sequence is provided below

SEQ ID NO: 23
ATGAAGTGGGTCACCTTCATTTCCTTGCTGTTCTTGTTCTCTTCCGCT
TACTCT

### Pre-Ost1-Alpha Signal Factor

In some embodiments pro region of alpha mating factor is fused with Pre-Ost1 and used as signal peptide

SEQ ID NO: 24
ATGAGGCAGGTTTGGTTCTCTTGGATTGTGGGATTGTTCCTATGTTTT
TTCAACGTGTCTTCTGCTGCTCCAGTCAACACTACAACAGAAGATGA
AACGGCACAAATTCCGGCTGAAGCTGTCATCGGTTACTCAGATTTA
GAAGGGGATTTCGATGTTGCTGTTTTGCCATTTTCCAACAGCACAAA
TAACGGGTTATTGTTTATAAATACTACTATTGCCAGCATTGCTGCTA
AAGAAGAAGGGGTATCTCTCGAGAAAAGAGAGGCTGAAGCT

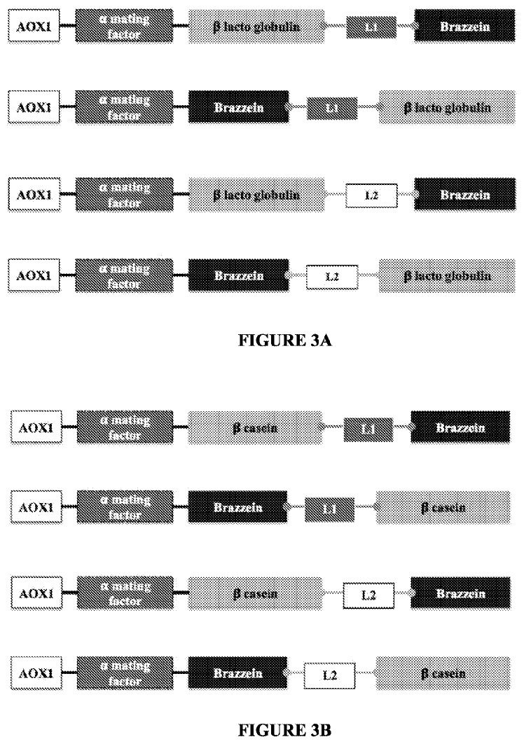

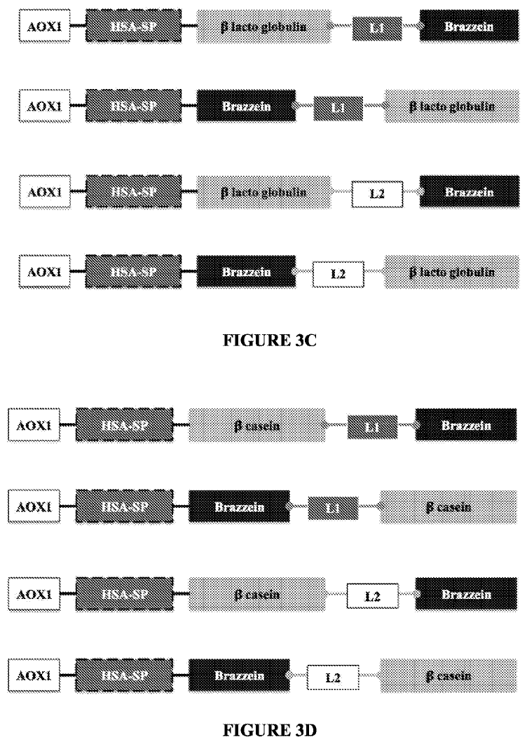

FIG. 3A-3D illustrates the various gene cassette representations, in accordance with an embodiment of the present invention.

FIG. 3A illustrates a gene cassette representation in which AOX1 is used as a vector and alpha mating signal factor is a signal peptide used in expression of recombinant proteins. The Beta lactoglobulin (Protein A) is linked with brazzein (Protein B) via “L1 type linker” and vice versa. The “L1 type linker” can be replaced by “L2 type linker” in same gene cassette.

FIG. 3B illustrates a gene cassette representation in which AOX1 is used as a vector and alpha mating signal factor is a signal peptide used in expression of recombinant proteins. The Beta casein (Protein A) is linked with brazzein (Protein B) via “L1 type linker “and vice versa. The “L1 type linker” can be replaced by “L2 type linker” in same gene cassette.

FIG. 3C illustrates a gene cassette representation in which AOX1 is used as a vector and Human Serum Albumin Signal Factor (HAS-SP) is a signal peptide used in expression of recombinant proteins. The Beta lactoglobulin (Protein A) is linked with brazzein (Protein B) via “L1 type linker” and vice versa. The “L1 type linker” can be replaced by “L2 type linker” in same gene cassette.

FIG. 3D illustrates a gene cassette representation in which AOX1 is used as a vector and Human Serum Albumin Signal Factor (HAS-SP) is a signal peptide used in expression of recombinant proteins. The Beta casein (Protein A) is linked with brazzein (Protein B) via “L1 type linker “and vice versa. The “L1 type linker” can be replaced by “L2 type linker” in the same gene cassette.

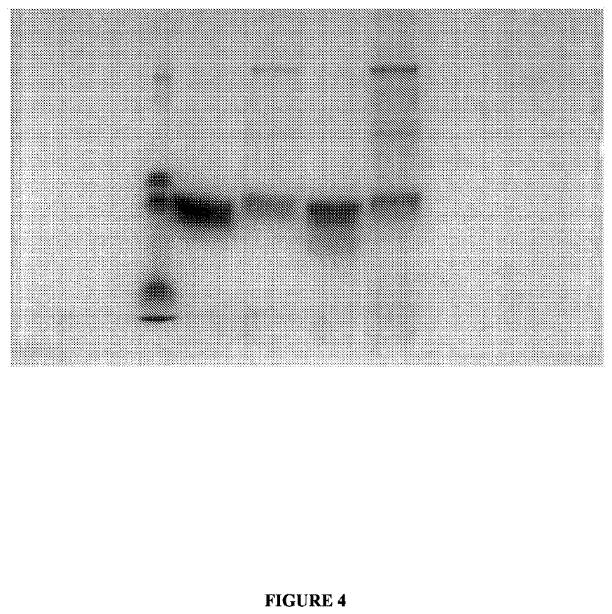

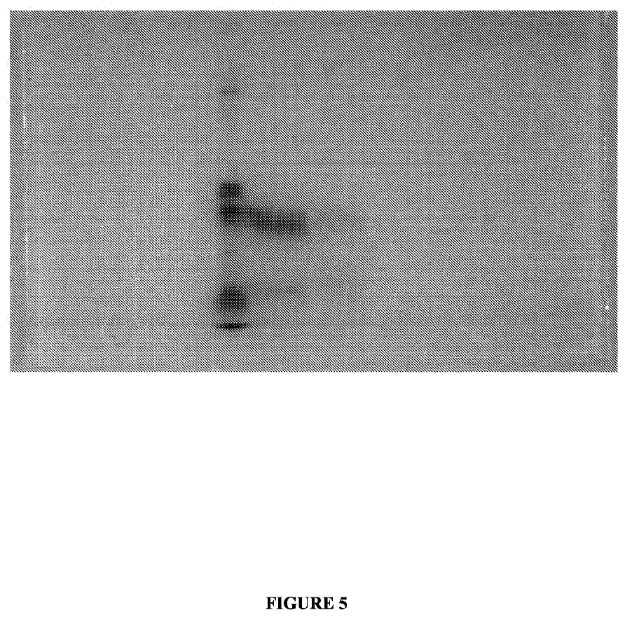

FIG. 4 shows the gel image for expression of Beta lactoglobulin-L1-Brazzein, Brazzein-L1-beta lactoglobulin, Beta lactoglobulin-L2-Brazzein, Brazzein-L2-beta lactoglobulin, in accordance with an embodiment of the present invention. In the FIG. 4, Lane 1 is blank, Lane 2 is blank, Lane 3 is of Milk protein standard, Lane 4 is of Beta lactoglobulin-L1-Brazzein, Lane 5 is of Brazzein-L1-Beta lactoglobulin, Lane 6 is of Beta lactoglobulin-L2-Brazzein, Lane 7 is Brazzein-L2-Beta lactoglobulin.

FIG. 5 shows the gel image for expression of Beta lactoglobulin-L3-Brazzein, Beta lactoglobulin-L4-Brazzein, in accordance with an embodiment of the present invention. In FIG. 5, Lane 1 is Blank, Lane 2 is Blank, Lane 3 is-Blank, Lane 4 is of Milk protein standard, Lane 5 is of Beta lactoglobulin-L3-Brazzein, Beta lactoglobulin-L4-Brazzein.

The present invention is further described in light of the following exemplary materials and methods which are set forth for an illustration purpose only and not to be construed for limiting the scope of the invention. The following experiments can be scaled up to industrial/commercial scale and the results obtained can be extrapolated to industrial scale. Additional materials and methods that can be used in any of the methods and compositions are also known in art.

### Compositions

In some embodiments the following compositions can be prepared using the recombinant fusion protein molecule. The non-limiting examples of the compositions are listed as—
1. Milk protein is linked to sweet proteins and combined with fats and ash, wherein the ash comprises essential minerals.
2. Non-milk protein is linked to sweet tasting proteins and combined with fats and ash, wherein the ash comprises essential minerals.
3. Milk protein and non-milk protein are linked to sweet tasting proteins and combined with fats and ash, wherein the ash comprises essential minerals.
4. Milk protein is linked to another milk protein.
5. Milk protein is linked to non-milk protein.
6. Non-milk protein is linked to non-milk protein.

### Proteins

In some embodiments the following proteins are used in the protein composition of the present invention. The non-limiting examples of the proteins are listed as:
1. Milk protein Kappa casein, alpha S1 casein, alpha S2 casein, Beta casein, beta lactoglobulin, alpha lactalbumin, transferrin and ovalbumin
2. Sweet proteins-Thaumatin, Monellin, Mabinlin, Miraculin, Curculin, Neoculin and Pentadin.

### Fats

In some embodiments the following fats are used in the composition of present invention. The non-limiting examples of the fats are listed as:
Plant based oils such as Sunflower oil, corn oil, soybean oil, palm fruit oil, palm kernel oil, safflower oil, flaxseed oil, rice bran oil, cotton seed oil, olive oil, canola oil, flaxseed oil, coconut oil; algal oil, microbial produced fats and fats derived from microbial fermentation of plant-based oil substrates.

### Ash

In some embodiments edible ash is used in the composition of the present invention.

### Minerals

The non-limiting examples of minerals are Calcium, Phosphorus and magnesium.

### Vectors

In the embodiments the following vectors are used in the protein composition of present invention. The non-limiting examples of the vectors are listed as:
1. Integrative vector containing methanol inducible promoter (AOX1, FLD1, MOX1, DAS1 etc), alpha mating signal factor, zeocin resistance cassette, terminator.
2. Integrative vector containing constitutive promoter, alpha mating signal factor, zeocin resistance cassette, terminator.
3. The vectors similar to vectors mentioned in the above points to 1 and 2 where zeocin is replaced with kanamycin resistance.
4. The vectors similar to vectors mentioned in the above points to 1-3 where alpha mating factor is replaced by any signal factor driving the expression to extracellular fraction.

### Expression Organisms

As per the embodiment of the present invention, the recombinant molecule in the above embodiments can be expressed in microbial systems used for recombinant molecules such as
Bacteria—Escherichia coli, Bacillus subtilis, Cyanobacteria etc
Fungal expression hosts—Aspergillus niger, Aspergillus nidulans, Trichoderma reesei, Pencillum sps
Yeasts—Saccharomyces cerevisiae, Candida albicans, Kluyveromyces lactis, Kluyveromyces marxianus, Yarrowia sps etc
Methylotrophic yeast—Pichia pastoris, Hansenula polymorpha, Candida boidini

### Transformation

Transformation protocols like chemical transformation using Lithium acetate and LiCl, transfection, electroporation, PEG mediated, agrobacterium mediated transformation and like can be used.

### EXAMPLES

The following examples are included to illustrate specific embodiments of this disclosure. The techniques disclosed in the examples represent techniques discovered by the inventors to function well in the methods and processes of this disclosure; however, those of skill in the art should, in light of the present disclosure, appreciate that many changes can be made in the specific embodiments that are disclosed and still obtain a like or similar result without departing from the spirit and scope of the disclosure. Therefore, all matter set forth or shown in the examples is to be interpreted as illustrative and not in a limiting sense.

### Recombinant Vectors

Briefed here is some of the exemplary recombinant vectors pPICZA-beta-lactoglobulin-L1-Brazzein, pPICZA-Brazzein-L1-beta-lactoglobulin, pPICZA-beta-lactoglobulin-L2-Brazzein, pPICZA-Brazzein-L2-beta-lactoglobulin, pPICZA-beta-lactoglobulin-L3-Brazzein, pPICZA-Brazzein-L3-beta-lactoglobulin, pPICZA-beta lactoglobulin-L4-Brazzein, pPICZA-Brazzein-L4-beta lactoglobulin, pPICZA-beta lactoglobulin-L1-Brazzein, pPICZA-beta lactoglobulin-L1-L1-Brazzein, pPICZA-beta lactoglobulin-L2-L2-Brazzein.

The gene of interest along with the chosen signal peptide was synthesized. The two genes were fused together using linker by overlapping PCR. The sequence for the linker was designed within the overlapping PCR primers. The fused construct was cloned at the EcoRI and NotI site of the pPICZA and transformed into E. coli NEB5 competent cells through the standard protocols. Transformants were selected on 25 μg/ml zeocin LB agar plates. The transformants were confirmed through restriction digestion and sequencing.

### Host Organisms

Pichia pastoris strain a derivative of BG 16 strain designated and referred to herein as PX-1.

### Strain Construction

The plasmids selected for Pichia transformation were isolated in large amounts through midi-prep. 40 μg of the plasmid DNA was linearized with SacI/PmeI enzyme and 5-10 μg of linearized vector was used to transform the above PX-1 strain through electroporation. The transformation protocol is briefly as follows. 100 ml YPD medium was inoculated with 20-50 ul inoculum of the PX-1 strain and grown overnight to an O.D of 1-1.3. The cells were harvested and washed twice with cold sterile milliQ water. In the next step they were washed with 2 ml of 1M sorbitol and finally resuspended in 200 ul of 1 M sorbitol. The cells were placed on ice and used the same day for electroporation. 80 ul of the competent cells were mixed with 5-10 μg of linearized DNA and electroporated using preset conditions for Pichia pastoris electroporation in Biorad electroporator. Immediately after electroporation 1 ml of 1M sorbitol was added and incubated for an hour at 30° C. Following that 1 ml YPD was added to the culture and further incubated for 2-3 hours at 30° C. while shaking. The colonies were selected on YPD-zeocin plates of varying concentration (100-2000 μg/ml). Integrated positive colonies were confirmed through colony PCR.

### Expression

Single colonies were inoculated in 10 ml BMGY media (1% yeast extract, 2% peptone, 100 mM potassium phosphate at pH 6.0, 1.34% Yeast Nitrogen Base, 4×10-5% biotin 1% glycerol) and cultured for 24 hrs and then transferred to BMMY (1% yeast extract, 2% peptone, 100 mM potassium phosphate at pH 6.0, 1.34% YNB, 4×10-5% biotin, 0.5% methanol) media to a final O.D of 1-2 for induction. After every 24 hrs 0.5% methanol was added to the cultures. Samples were collected at 24, 48 and 72 hrs and were visualized on the SDS page for expression. Positive samples were then serially scaled to 500 ml flask, 1 ml flask and 8 L fermentor.

### Fermenter Conditions

Glycerol stock of the strain was inoculated into 50 mL of YPD broth in a 250 mL of baffled flask. This inoculum was then used to inoculate 100 ml of GYP (Yeast extract 1%, Peptone 2% and Glycerol 2%) as adaptation stage. This was then inoculated in 250 ml of BSM (Glycerol 4%, Orthophosphoric acid 2.7%, K2SO41.8%, MgSO4.7H2O 1.4%, KOH 0.413%, CaSO4.2H2O 0.116%). The culture was then used to inoculate a 8 L ferementor containing BSM media, maintaining pH at 5 for growth phase and 6 for induction phase. Temperature was maintained at 30 for growth phase and 24 for induction phase.

### Protein Purification

The culture supernatant was initially processed through microfiltration to remove cells and other particulate matter. The permeate was then concentrated using ultrafiltration/diafiltration followed by freeze drying to obtain powdered protein.

### Taste Testing

A double-blind study was conducted using 20 participants to analyze the sweetness quotient of the samples. Sucrose solution of 30 g/L at 20 C was taken as a reference solution and was given a sweetness quotient of one. Participants were given the samples to taste and asked to rate the sweetness quotient based on sucrose standard as 1.

### Example 1: Construction of Recombinant Vectors for Expression of Recombinant Fusion Proteins

Briefed here is some of the exemplary recombinant vectors pPICZA-beta-lactoglobulin-L1-Brazzein, pPICZA-Brazzein-L1-beta-lactoglobulin, pPICZA-beta-lactoglobulin-L2-Brazzein, pPICZA-Brazzein-L2-beta-lactoglobulin, pPICZA-beta-lactoglobulin-L3-Brazzein, pPICZA-Brazzein-L3-beta-lactoglobulin, pPICZA-beta lactoglobulin-L4-Brazzein, pPICZA-Brazzein-L4-beta lactoglobulin, pPICZA-beta lactoglobulin-L1-Brazzein, pPICZA-beta lactoglobulin-L1-L1-Brazzein, pPICZA-beta lactoglobulin-L2-L2-Brazzein. The gene of interest along with the chosen signal peptide was synthesized. The two genes were fused together using linker by overlapping PCR. The sequence for the linker was designed within the overlapping PCR primers. The fused construct was cloned at the EcoRI and NotI site of the pPICZA and transformed into E. coli NEB5 competent cells through the standard protocols. Transformants were selected on 25 μg/ml zeocin LB agar plates. The transformants were confirmed through restriction digestion and sequencing.

### Host Organisms

Pichia pastoris strain—A derivative strain of BG16, procured from Atum Biosciences.

Strain construction—The plasmids selected for Pichia transformation were isolated in large amounts through midi prep protocol. 40 μg of the plasmid DNA was linearized with SacI/PmeI enzyme and 5-10 μg of linearized vector was used to transform the above PPS-9016 strain through electroporation. The transformation protocol is briefly as follows. 100 ml YPD medium was inoculated with 20-50 ul inoculum of the PX-1 strain and grown overnight to an O.D of 1-1.3. The cells were harvested and washed twice with cold sterile milliQ water. In the next step they were washed with 2 ml of 1M sorbitol and finally resuspended in 200 ul of 1 M sorbitol. The cells were placed on ice and used the same day for electroporation. 80 ul of the competent cells were mixed with 5-10 μg of linearized DNA and electroporated using present conditions for Pichia pastoris electroporation in Biorad electroporator. Immediately after electroporation 1 ml of 1M sorbitol was added and incubated for an hour at 30° C. Following that 1 ml YPD was added to the culture and further incubated for 2-3 hours at 30° C. while shaking. The colonies were selected on YPD-zeocin plates of varying concentration (100-2000 μg/ml). Integrated positive colonies were confirmed through colony PCR.

### Example 2: Expression of the Recombinant Fusion Protein

Single colonies were inoculated in 10 ml BMGY media (1% yeast extract, 2% peptone, 100 mM potassium phosphate at pH 6.0, 1.34% Yeast Nitrogen Base, 4×10-5% biotin 1% glycerol) and cultured for 24 hrs and then transferred to BMMY (1% yeast extract, 2% peptone, 100 mM potassium phosphate at pH 6.0, 1.34% YNB, 4×10-5% biotin, 0.5% methanol) media to a final O.D of 1-2 for induction. After every 24 hrs 0.5% methanol was added to the cultures. Samples were collected at 24, 48 and 72 hrs and were visualized on the SDS page for expression. Positive samples were then serially scaled to 500 ml flask, 1 ml flask and 8 L fermenter.

Fermenter conditions: Glycerol stock of the strain was inoculated into 50 mL of YPD broth in a 250 mL of baffled flask. This inoculum was then used to inoculate 100 ml of GYP (Yeast extract 1%, Peptone 2% and Glycerol 2%) as adaptation stage. This was then inoculated in 250 ml of BSM (Glycerol 4%, Orthophosphoric acid 2.7%, K2SO41.8%, MgSO4.7H2O 1.4%, KOH 0.413%, CaSO4.2H2O 0.116%). The culture was then used to inoculate an 8 L fermenter containing BSM media, maintaining pH at 5 for growth phase and 6 for induction phase. Temperature was maintained at 30° C. for growth phase and 24° C. for induction phase.

### Example 3: Isolation and Purification of Fusion Protein

The culture supernatant was initially processed through microfiltration to remove cells and other particulate matter. The permeate was then concentrated using ultrafiltration/diafiltration and freeze dried to obtain protein powder.

### Example 4: Determination of Palatability of the Recombinant Fusion Protein

Taste testing—A double blind study was conducted using 20 participants to analyse the sweetness quotient of the samples. Sucrose solution of 30 g/L at 20 C was taken as a reference solution and was given a sweetness quotient of one. Participants were given the samples to taste and asked to rate the sweetness quotient based on sucrose standard as 1.

The above descriptions are only the preferred embodiments of the present invention and are not intended to limit the present invention. Any modification, equivalent replacement, improvement, etc. made within the spirit and principle of the present invention shall be included within the scope of the present invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the disclosure to achieve one or more of the desired objects or results.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1. A recombinant fusion protein comprising:
a.) at least a first protein comprising an amino acid sequence having at least 80% sequence identity to the polypeptides as encoded by a nucleotide sequence selected from SEQ ID NO: 1, SEQ ID NO: 2, SEQ ID NO: 3. SEQ ID NO: 4, SEQ ID NO: 5 SEQ ID NO: 6, and SEQ ID NO: 7;
b.) at least a second protein comprising an amino acid sequence having at least 80% sequence identity to the polypeptides as encoded by a nucleotide sequence selected from the group consisting of SEQ ID NO: 8, SEQ ID NO: 9, SEQ ID NO.: 10, SEQ ID NO.: 11, SEQ ID NO.: 12, and SEQ ID NO.: 13; and
c.) at least a linker comprising an amino acid sequence coded by nucleotide sequence which is at least 80% similar to sequences selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21.

2. The recombinant fusion protein of claim 1, wherein the first protein is selected from the group comprising of a milk protein, a non-milk bovine protein, and a non-bovine protein of microbial, animal and plant origin.

3. The recombinant fusion protein of claim 2, wherein the milk protein is a recombinantly expressed protein selected from the group comprising of alpha-lactalbumin, beta-lactoglobulin, kappa-casein, alpha-casein, beta-casein, lactoferrin and the like.

4. The recombinant fusion protein of claim 2, wherein the milk protein is a recombinant beta-lactoglobulin.

5. The recombinant fusion protein of claim 1, wherein the second protein is selected from the group comprising of a low-calorie non-carbohydrate sweetener or a non-milk bovine protein or a non-bovine protein of microbial, animal, or plant origin.

6. The recombinant fusion protein of claim 5, wherein the low-calorie non-carbohydrate sweetener is selected from the group consisting of Brazzein, Thaumatin, Monellin, Curculin, Mabinlin, Miraculin, Neoculin, Pentadin and a combination thereof.

7. The recombinant fusion protein of claim 6, wherein the low-calorie non-carbohydrate sweetener is Brazzein.

8. The recombinant fusion protein of claim 1, wherein the first protein is fused with the second protein using an inert peptide linker comprising amino acids coded by the nucleotide sequence which is at least 80% similar to sequences selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21.

9. The recombinant fusion protein of claim 1 wherein the orientation of first protein and second protein are interchangeable.

10. An expression vector, comprising the nucleic acid molecule encoding the recombinant fusion protein claim 1.

11. A composition comprising:
a) at least one recombinant fusion protein:
b) at least one non-fusion protein; and
c) at least one additive,
wherein the at least one recombinant fusion protein comprises:
at least a first protein comprising an amino acid sequence having at least 80% sequence identity to the polypeptides as encoded by a nucleotide sequence selected from SEQ ID NO: 1, SEQ ID NO: 2, SEQ ID NO: 3. SEQ ID NO: 4, SEQ ID NO: 5 SEQ ID NO: 6, and SEQ ID NO: 7;
at least a second protein comprising an amino acid sequence having at least 80% sequence identity to the polypeptides as encoded by a nucleotide sequence selected from the group consisting of SEQ ID NO: 8, SEQ ID NO: 9, SEQ ID NO.: 10, SEQ ID NO.: 11, SEQ ID NO.: 12, and SEQ ID NO.: 13; and
at least a linker having an amino acid sequence coded by nucleotide sequence which is at least 80% similar to sequences selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, and SEQ ID NO: 21.

12. The composition of claim 11, wherein,
a) said at least one recombinant fusion protein being in the range of 0.1% to 8%;
b) said at least one non-fusion protein in the range of 1% to 10%; and
c) said at least one additive in the range of 0.1% to 5%.

13. The composition of claim 11, wherein the at least one non-fusion protein is selected from the group consisting of microbial, animal, plant origin or recombinantly expressed protein.

14. The composition of claim 11, wherein the first protein of the recombinant fusion protein is selected from the group comprising of a milk protein, a non-milk bovine protein, and a non-bovine protein.

15. The composition of claim 14, wherein the milk protein of the recombinant fusion protein is selected from the group comprising of alpha-lactalbumin, beta-lactoglobulin, kappa-casein, alpha-casein, beta-casein, lactoferrin and the like.

16. The composition of claim 11, wherein the second protein is selected from the group comprising of a low-calorie non-carbohydrate sweet protein or a non-milk bovine protein or a non-bovine protein of microbial, animal, plant origin or recombinantly expressed protein.

17. The composition of claim 16, wherein the low-calorie non-carbohydrate sweet protein is selected from the group consisting of Brazzein, Thaumatin, Monellin, Curculin, Mabinlin, Miraculin, Neoculin, Pentadin and a combination thereof.

18. The composition of claim 11, wherein the at least one additive is selected from the group consisting of lipids, fats, ash, micronutrients, vitamins, water, and the like.

19. The composition of claim 11, wherein the recombinant fusion protein or the composition is used in formulating dairy and related products.

20. (canceled)
